(19) United States
(12) Patent Application Publication
(10) Pub. No.: US 2006/0161260 A1
(43) Pub. Date: Jul. 20, 2006

(54) Total wrist prosthesis

(76) Inventors: Gareth Thomas, Dorset (GB); John Knowles Stanley, Lancashire (GB)

Correspondence Address: BAKER BOTTS L.L.P., 2001 ROSS AVENUE, SUITE 600, DALLAS, TX, 75201-2980, US

(21) Appl. No.: 10/746,982

(22) Filed: Dec. 23, 2003

(51) Int. Cl. A61F 2/42

(52) U.S. Cl. 623/21.12

(57) ABSTRACT

A prosthesis includes an aperture with an arcuate wall incorporating a locking bone screw having an internally threaded annular collar with a circumferential cut-out allowing the collar to expand and contract. The collar may have an arcuate outer surface to engage and rotate in an arcuate wall of an aperture. The screw may include a head at one end of a screw shank, a first thread on the screw shank extending from the screw end remote from the head towards the head. A second thread on the shank may be adjacent the head. The screw may also include a threadless section of shank or lag between the first and second thread. The shank supporting the second thread may be tapered such that it expands the collar when screwed into the collar.

### TECHNICAL FIELD OF THE INVENTION

The present invention relates to a total wrist prosthesis.

### BACKGROUND OF THE INVENTION

Various wrist prosthesis have hitherto been known. Most of these allow for movement into two planes namely flexion and extension movement in a first plane, and ulnar deviation and radial deviation in another plane. However these do not allow for small rotational movements of the hand relative to the arm in a third plane. Also some prosthesis parts are joined too loosely leading to disjointing problems.

### SUMMARY OF THE INVENTION

The invention seeks to provide a prosthesis which allows hand movements in three planes and which gives a reliable joint, reducing the chances of dislocation and/or loosening.

According to a particular embodiment of the present invention there is provided a total wrist prosthesis comprising;

a) a proximal radial component having a radial body and a stem extending from one side of the body that allows fixation into the distal radius,

b) a distal carpal component having a carpal body and one or more stems extending from one side of the body that allows fixation into the capitate, and

c) an intermediate component, said intermediate component being connected to said carpal component so as to allow rotational movement of the carpal component relative to the intermediate component whereby to allow small rotational movements of the hand relative to the arm, and said intermediate component being connected to the radial component so as to allow only vertical and horizontal hinged movement between the intermediate and radial component so as to allow for flexion and extension movement in a first plane and lunar and radiation movement in a second plane.

In one embodiment, the carpal component has one or more apertures to receive screws for fixation into one or more carpals.

In one embodiment, the radial body has a toroidal concave recess or convex projection on its side remote from its shaft to engage with a toroidal convex projection or concave recess on the intermediate component.

In one embodiment the radial body has an toroidal concave recess to engage with a toroidal convex projection on the intermediate component. The carpal body may have a projection on its side remote from its shafts which rotates in an aperture in the intermediate component. In one embodiment the projection is generally cylindrical having a pair of opposed lugs extending from the cylindrical walls thereof, said projections being engaged in an annular groove in the intermediate component aperture to prevent separation of the carpal component from the intermediate component. The projections may slide through a side keyway in the intermediate component and rotate in the groove to assemble the intermediate component and carpal component together.

### BRIEF DESCRIPTION OF THE DRAWINGS

An embodiment of the invention will now be described with reference to the accompanying drawings in which:

FIGS. 1A and 1B show a perspective exploded view of a prosthesis from either end,

FIG. 2 shows a side view of FIG. 1,

FIG. 3 shows an end view of FIG. 1,

FIG. 4 shows a collar and screw in an aperture of part of a prosthesis,

FIG. 5 shows a cross section and end view of a collar, and

FIG. 6 shows a side and end view of the screw.

### DETAILED DESCRIPTION OF THE DRAWINGS

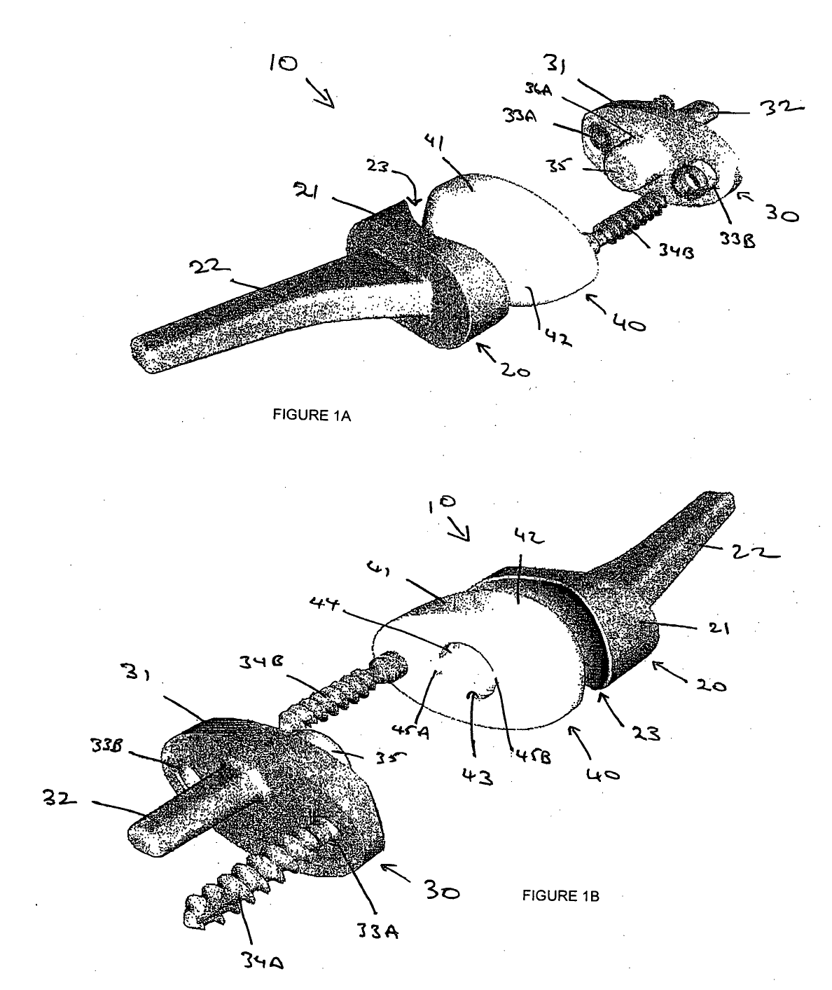

Referring to FIGS. 1 to 3 there is shown a prosthesis 10. Prosthesis 10 has a proximal radial component 20, a distal carpal component 30, and an intermediate component 40.

Proximal radial component 20 may be formed from any suitable material such as a cobalt chrome alloy. Component 20 has a generally oval body 21, and a stem 22 extending from one side of the body that allows fixation into the distal radius. Part of the body and/or shaft may be coated with a textured bone ingrowth surface to provide a key for the bone.

Radial body 21 has a toroidal concave recess 23 on its side remote from its stem 22. The recess 23 has the largest radius of curvature over the body length and the smallest radius of curvature over the body width normal to the largest radius of curvature. Also as shown in dotted lines, one side and end wall of the body may be higher than its opposite side and end wall, e.g. to provide a 10 degree slope across the two side and two end walls, to reduce subluxion when the prosthesis is used.

Distal carpal component 30 may be formed from any suitable material such as a cobalt chrome alloy. Component 30 has a generally oval carpal body 31 and stem 32 extending from one side of the body 31 that facilitates location into the capitate. A pair of apertures 33A, 33B are provided either side of stem 32 to receive screws to provide stabilisation and fixation into the remaining carpal bones. Such screws may be locking screws e.g., of the type described later, in more detail. Accordingly apertures 33A, 33B may be adapted to receive screws with collars 34A 34B of the type described and shown herein. Again, part of the body and/or shafts may be coated with a textured bone ingrowth surface to provide a key for the bone.

Carpal body 31 has a cylindrical projection 35 on its side remote from its stem 32 having a pair of opposed lugs 36A, 36B extending from the cylindrical walls thereof.

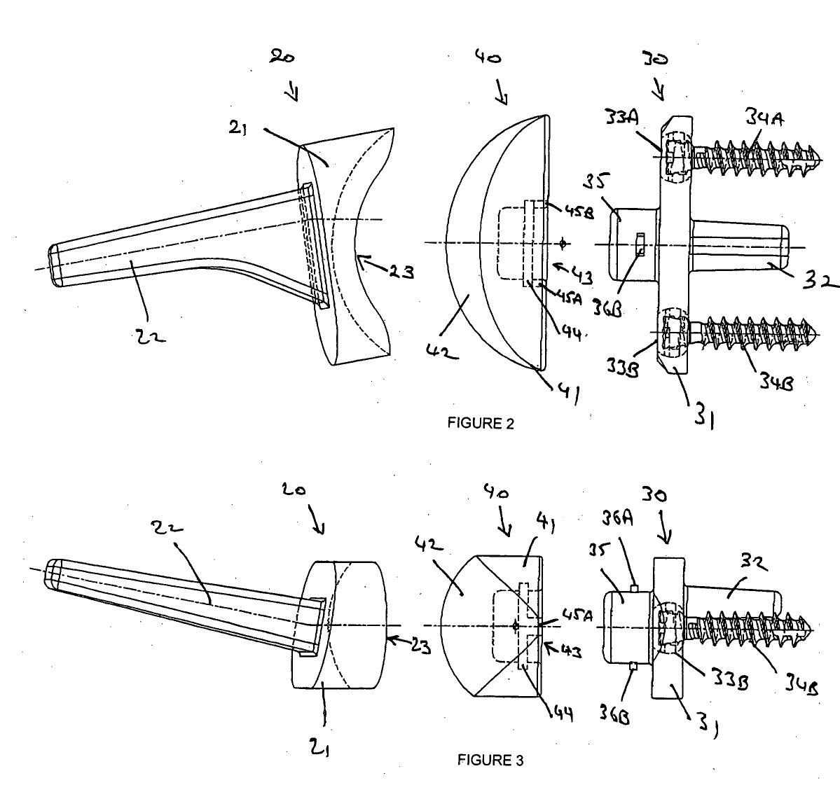

Intermediate component 40 has an oval body 41 formed from ultra high molecular weight polyethylene to provide a low motion bearing surface against cobalt chrome. One side of body 41 has a toroidal projection 42. As shown in FIGS. 2 and 3, the projection has the largest radius of curvature over the body length and the smallest radius of curvature over the body width normal to the largest radius of curvature and these correspond to substantially the same radii of curvature of the recess 23 in radial body 21.

In use, the ellipsoidal projection 42 and mating ellipsoidal recess 23 allow only vertical and horizontal hinged movement between the intermediate and radial component so as to allow for flexion and extension movement in a first plane and lunar and radiation movement in a second plane.

On the opposite side of body 41 to projection 42 is a cylindrical aperture 43 having an annular groove 44 therein. Grooves 45A, 45B provide a keyway from the open end of aperture 43 into the annular groove 44. Carpal body 31 can be releasably connected to the intermediate component 40 by pushing the cylindrical projection 35 into cylindrical aperture 43 with lugs 36A, 36B sliding in grooves 45A, 45B, and then the body 41 is rotated through 90 degrees so that lugs 36A, 36B enter annular groove 44. With lugs 36A, 36B in annular groove 44, separation of the carpal component from the intermediate component is prevented. In use, this allows rotational movement of the carpal component relative to the intermediate component whereby to allow small rotational movements of the hand relative to the arm.

The invention may take a form other than that specifically described above. For example the proximal component may be connected and/or coupled to the intermediate component by another method. For example the toroidal (or other shape) recess could be on the intermediate component and the toroidal projection on the proximal component. Also other types of joints could be used to allow the distal component to rotate relative to the intermediate component.

Angling of the shaft 22 provides good anatomical compliance and supports the prosthesis during flexion and extension to prevent volar dislocation. Also the toroidal projection and recess on the intermediate and proximal component respectively is designed to mimic anatomic movement.

The teachings of the present invention may also include a locking screw, especially for securing a medical prosthesis to a bone.

Locking bone screws may be used to secure prostheses to bones. Such locking screws have two components. One component is an internally-threaded annular collar with a circumferential cut out allowing the collar to expand and contract. The collar has an arcuate outer surface to engage and rotate in an arcuate wall of an aperture in a prosthesis. The other component is a screw having a continuous and constant thread extending from a screw shank which tapers outwards towards the screw head. In use the collar is inserted in the prosthesis and the screw placed into threaded engagement with the collar. The screw can then be screwed into the bone. Before the screw is screwed fully into the bone and collar, the screw can toggle relative to the prosthesis so its angle can change as the collar can rotate in the aperture of the prosthesis. However when the screw is fully screwed into the bone and collar, the widened taper of the shank of the screw expands the collars so locking the collar in the aperture and the screw in the collar.

A problem with this known arrangement is that an ideal screw thread for engaging bone tissue is not always the ideal screw thread for engaging the collar.

The teachings of the present invention seek to provide an improved locking screw, especially for bones.

According to the present invention there is provided a locking screw comprising:

a) an internally-threaded annular collar with a circumferential cut out allowing the collar to expand and contract, said collar having an arcuate outer surface to engage and rotate in an arcuate wall of an aperture;

b) a screw having a head at one end of a screw shank, a first thread on the screw shank extending from the screw end remote from the head towards the head, a second thread on the shank adjacent the head, and a threadless section of shank or lag between the first and second thread, said shank supporting the second thread being tapered such that it expands the collar when screwed into the collar.

The locking screw may be a locking bone screw.

The first thread may be a cancellous thread such as an ISO standard bone thread.

The second thread may be a machine thread such as an ISO machine thread.

The invention also extends to a prosthesis with an aperture with an arcuate wall incorporating the above defined locking bone screw. The aperture may have an annular flange at one end of the aperture to prevent the collar passing through said end of the aperture.

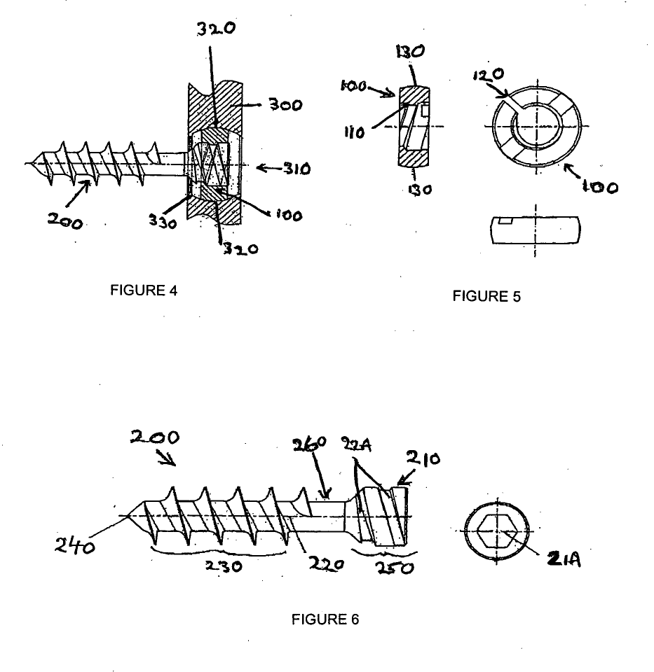

An embodiment of the locking screw is described with reference to FIGS. 4-6.

Referring to FIG. 4 there is provided a locking bone screw having an annular collar 100 and screw 200 in an aperture 310 of a prosthesis 300.

Referring more particularly to FIG. 5, annular collar 100 has an internal thread 110. A circumferential cut out 120 is provided allowing the collar to expand and contract. Collar 100 may be formed of resilient material such as metal, e.g. titanium-based metal. Collar 100 has an arcuate outer surface 130 to engage and rotate in an arcuate wall 320 of aperture 310 in prosthesis 300.

As shown in FIG. 6, screw 200 has a head 210 at one end of a screw shank 220. Screw 200 may be of metal such as titanium-based metal. Screw head may have a hexagonal recess 21A to receive a tool (not shown) to rotate the screw.

A first thread 230 on the screw shank extends from the screw end 240 remote from the head 210 towards the head 210. First thread 230 is a cancellous thread such as an ISO standard bone thread.

A second thread 250 is provided on the shank 220 adjacent the head. The shank section 22A supporting thread 250 tapers outwards toward the end of the head 210. The second thread 250 is a machine thread such as an ISO machine thread.

A threadless section or lag 260 of the shank is provided between the first and second thread.

First thread 230 and second thread 250 are of a size to engage with the internal thread 110 of collar 100. However when first thread 230 is engaged with internals thread 110 of collar 100, collar 100 is able to rotate in aperture 310 allowing the screw 200 to toggle relative to the prosthesis. When second thread 250 is fully engaged with internal thread 110 (e.g. the screw 200 is fully screwed into collar 100 as shown in FIG. 4), the tapered shaft supporting thread 250 expands collar 100 so its arcuate outer surface 130 locks against the arcuate wall 320 of aperture 310 in prosthesis 300.

It is preferred that an annular flange 330 is provided at one end of the aperture 310 to prevent the collar passing through this end of the aperture, so preventing separation of the screw 200 from the prosthesis 300. Flange 330 also limits rotation of the collar 100 in aperture 310.

In use screw 200 is placed in collar 100 in the prosthesis aperture 310, and is screwed into a bone using a tool with the thread 230 engaging the bone tissue. The desired angle of the screw is achieved as it can toggle relative to the prosthesis. The collar 100 finally locks in the aperture 310 as the head 210 is screwed into the collar.

Because the thread 250 is a machine thread it engages well with the metal collar to prevent it unscrewing itself, and the bone thread 230 bonds well with bone.

Once the prosthesis is connected to the bone, after a period of time, fibrous growth forms around the threadless section or lag in shank 220 which helps prevent the screw from unscrewing out of the bone.

The invention may take a form different to that specifically described above.

The locking screw of the invention may be sold separately or in conjunction with a prosthesis. The locking screw of the invention may be of particular use when used with the wrist prosthesis as defined herein.

The locking bone screw may have applications outside medical application, i.e. used to secure fittings to material other than bones.

Further modifications will be apparent to those skilled in the art without department from the scope of the present invention.

What is claimed is:

1. A locking screw comprising:
a) an internally-threaded annular collar with a circumferential cut out allowing the collar to expand and contract, said collar having an arcuate outer surface to engage and rotate in an arcuate wall of an aperture,
b) a screw having a head at one end of a screw shank, a first thread on the screw shank extending from the screw end remote from the head towards the head, a second thread on the shank adjacent the head, and a threadless section of shank or lag between the first and second thread, said shank supporting the second thread being tapered such that it expands the collar when screwed into the collar.

2. A locking screw according to claim 1, wherein the locking screw is a locking bone screw.

3. A locking screw according to claim 1 or 2, wherein the first thread is a cancellous thread such as an ISO standard bone thread.

4. A locking screw according to claim 1, 2 or 3, wherein the second thread is a machine thread such as an ISO machine thread.

5. A prosthesis with an aperture with an arcuate wall incorporating a locking bone screw according to claim 1, 2, 3 or 4.

6. A prosthesis according to claim 5, wherein the aperture has an annular flange at one end of the aperture to prevent the collar passing through said end of the aperture.
